(19) United States
(12) Patent Application Publication
(10) Pub. No.: US 2010/0157248 A1
(43) Pub. Date: Jun. 24, 2010

(54) COLOR TINTING OF LIGHT IN OPHTHALMIC FIBER OPTIC ILLUMINATION SYSTEMS

(76) Inventor: R. A. Edwards, Burwell (GB)

Correspondence Address: Bausch & Lomb Incorporated, One Bausch & Lomb Place, Rochester, NY, 14604-2701, US

(21) Appl. No.: 12/341,450

(22) Filed: Dec. 22, 2008

(51) Int. Cl. A61B 3/00

(52) U.S. Cl. 351/221; 351/246

(57) ABSTRACT

A method is described for providing color-tinted light in an ophthalmic illumination system having a light source that produces non-white light. A color tint for illuminating one or more ophthalmic features of interest is selected. Chromaticity coordinates for the non-white light are determined. The method includes determining, based on the determined chromaticity coordinates, a spectral region to be blocked from the non-white light to obtain light from the light source exhibiting the selected color tint. The illumination system is provided with one or more filters configured to block the spectral region from the non-white light.

### FIELD

The present disclosure relates to ophthalmic illumination systems and more particularly to color tinting of light in an ophthalmic fiber optic illumination system.

### BACKGROUND

This section provides background information related to the present disclosure which is not necessarily prior art.

When ophthalmic surgery is performed, an ophthalmic illumination system is used to illuminate the interior of a patients eye so that the surgeon may view the surgical site. In a typical ophthalmic illumination system, light is collimated and focused onto an entrance pupil of optical fiber connected to an opto-illuminator, or light probe. A tip of the probe is inserted into an incision in the eye.

### SUMMARY

This section provides a general summary of the disclosure, and is not a comprehensive disclosure of its full scope or all of its features.

In one configuration, the present disclosure is directed to a method of providing color-tinted light in an ophthalmic illumination system having a light source configured to produce non-white light. A color tint for illuminating one or more ophthalmic features of interest is selected. Chromaticity coordinates for the non-white light are determined. The method includes determining, based on the determined chromaticity coordinates, a spectral region to be blocked from the non-white light to obtain light from the light source exhibiting the selected color tint. The illumination system is provided with one or more filters configured to block the spectral region from the non-white light.

In another implementation, the disclosure is directed to a method of providing color-tinted light in an ophthalmic illumination system having a xenon light source configured to produce safety-filtered light. A color tint for illuminating one or more ophthalmic features of interest is selected and chromaticity coordinates for the selected color tint are determined. Chromaticity coordinates for the safety-filtered light are determined. The determined coordinates are used to locate a spectral region to be blocked from the safety-filtered light to obtain light exhibiting the selected color tint. The illumination system is provided with one or more filters configured to filter frequencies in the located spectral region from the safety-filtered light.

In yet another implementation, the disclosure is directed to an ophthalmic illumination system having a light source and a light probe configured to receive light from the light source via an optical light path. The illumination system includes one or more filters configured to cause non-white light from the light source to exhibit a selected color tint. At least one of the filter(s) is configured to block from the non-white light a spectral region away from which a chromaticity of the non-white light is changeable to cause the light to exhibit a chromaticity of the selected tint.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a diagrammatic representation of an ophthalmic illumination system in accordance with one implementation of the disclosure;

FIG. 2 is a color space chromaticity diagram;

FIG. 3 is a portion of the color space chromaticity diagram shown in FIG. 2, on which are indicated xy coordinates for chromaticity values in accordance with one implementation of the disclosure;

FIG. 4 is a graph of a transmission curve for a blocking region to provide a yellow tint for safety-filtered xenon light in accordance with one implementation of the disclosure;

FIG. 5 is a graph of a transmission curve for a blocking region to provide a cyan tint for safety-filtered xenon light in accordance with one implementation of the disclosure;

FIG. 6 is a graph of a transmission curve for a blocking region to provide a green tint for safety-filtered xenon light in accordance with one implementation of the disclosure; and

FIG. 7 is a flow diagram of a method of providing color-tinted light in an ophthalmic illumination system in accordance with one implementation of the disclosure.

Corresponding reference numerals indicate corresponding parts throughout the several views of the drawings.

### DETAILED DESCRIPTION

Example embodiments will now be described more fully with reference to the accompanying drawings.

It has been observed that eye surgeons can obtain improved visualization of various eye features by using illumination systems in which light from a nominally white lamp source, e.g., a metal halide lamp, is filtered to exhibit various low-saturation colors, Le., color tints, White light that is yellow-tinted has been observed to provide improved viewing of optic discs, blood vessels, vitreous, and retinas. Green-tinted and cyan-tinted light have each been shown to provide improved viewing for some if not all of the foregoing structures.

Different types of light sources, however, typically have different spectral distributions. For example, although the visible part of the spectrum of a xenon lamp gives a substantially white light, when xenon lamp light is filtered for safety reasons the light becomes somewhat green. When inserted into a white light beam, a “yellow” filter may provide a desired yellow tint. Such a filter, however, generally would not achieve the same results when used with a non-white light source. In various implementations of the disclosure, non-white light is filtered to cause the light to exhibit a selected color tint. A surgeon can use such filtering to selectively change the color tint of non-white light from, e.g., a safety-filtered xenon lamp during surgery to optimize visibility for a particular eye feature being examined.

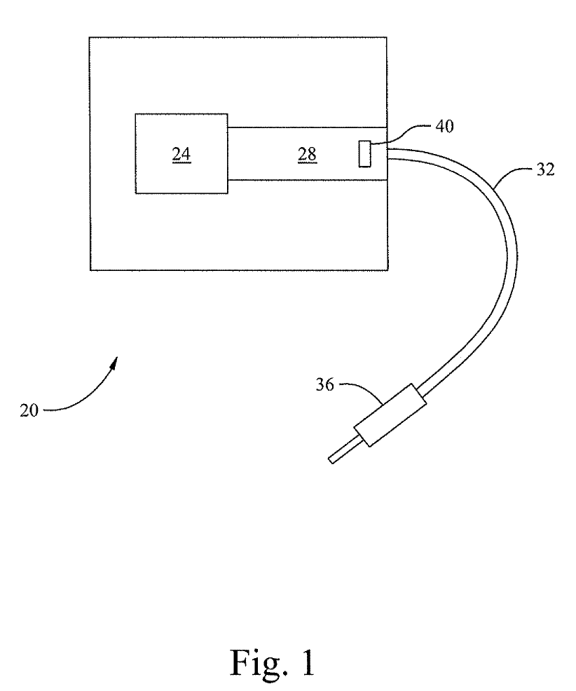

A diagrammatic representation of an ophthalmic illumination system in accordance with one implementation of the disclosure is indicated generally in FIG. 1 by reference number 20. A light source 24 provides light that is collected, collimated, and refocused via light collection optics 28 for transmission through an optical fiber 32 to a light probe 36. In the present exemplary embodiment the light source 24 is a xenon lamp. It should be noted, however, that in other implementations of the disclosure other types of non-white light sources could be used.

The xenon lamp 24 is “safety-filtered”. That is, selected portions of the ends of the spectrum of light from the lamp 24 are filtered out for safety reasons. Specifically and for example, ultraviolet (UV) wavelengths (less than about 400 nanometers) and the deep blue part of the visible spectrum (wavelengths between about 400 nanometers and about 450 nanometers) are filtered out of the xenon lamp light. Infrared (IR) wavelengths (greater than about 700 nanometers) and the deep red part of the visible spectrum (wavelengths between about 650 nanometers and about 700 nanometers) are also filtered out of the xenon lamp light for safety reasons. Filtering out UV and IR wavelengths has no effect on the visible color of xenon lamp light. However, filtering out the deep blue and deep red parts of the visible spectrum results in the color of safety-filtered light from the xenon lamp 24 becoming somewhat green.

One or more filters 40 are selectable, e.g., by a surgeon using the system 20, to color-tint the light being transmitted from the light source 24 to the light probe 36. As further described below, a filter 40 provides color tinting by blocking from the non-white light a spectral region away from which a chromaticity of the non-white light is changeable to cause the light to exhibit a chromaticity of the selected tint.

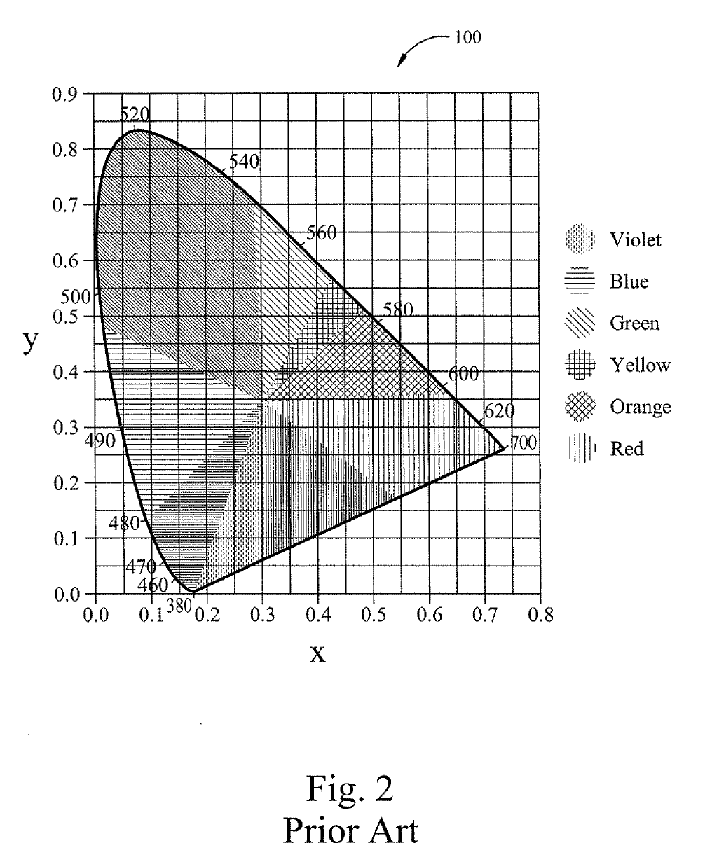

Various implementations of the disclosure are described with reference to a color space chromaticity diagram indicated generally in FIG. 2 by reference number 100. The diagram 100 is an approximation of the 1931 International Commission on Illumination (CIE) xy chromaticity diagram. FIG. 2 uses guidelines from the United States Patent Office to indicate, generally, areas of different colors. It should be noted, however, the disclosure could be implemented with reference to other chromaticity diagrams that represent chromaticity as coordinates. CIE xy coordinates shown in Table 1 are used to represent chromaticity for one set of tints that has been observed to be preferred by a group of eye surgeons.

TABLE 1

| | CIE x | CIE y |
|---|---|---|
| Yellow | 0.3534 | 0.3619 |
| Cyan | 0.2462 | 0.2929 |
| Green | 0.2954 | 0.3901 |

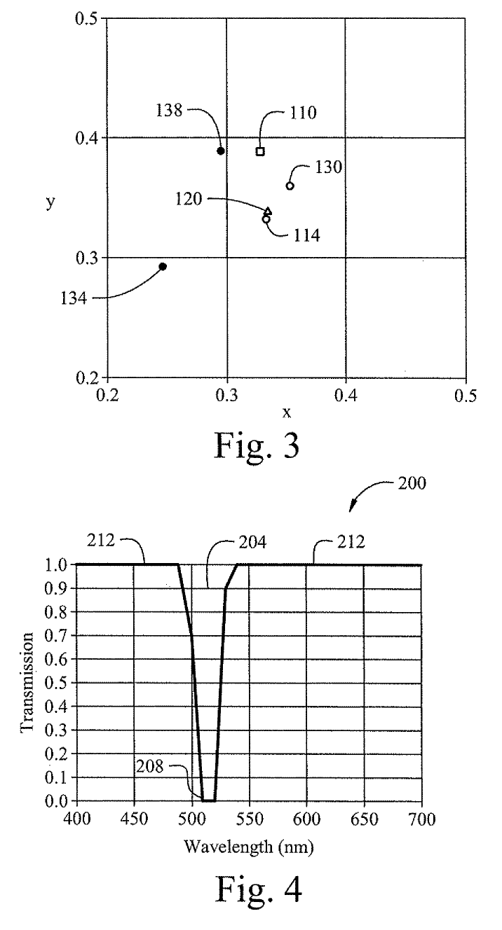

CIE xy values may be used to represent a spectral distribution as a single point giving an indication of perceived color. As previously discussed, a safety-filtered xenon lamp such as the lamp 24 provides light that exhibits a somewhat greenish hue. A portion of the diagram 100 is shown in FIG. 3. A square 110 represents chromaticity for light from a safety-filtered xenon lamp. A circle 114 represents chromaticity for white. A triangle 120 represents the chromaticity of light from a metal halide lamp, which is close to the chromaticity 114 for white. Circles 130, 134 and 138 represent chromaticity for the set of tints shown in Table 1. Specifically, the circle 130 represents yellow, the circle 134 represents cyan, and the circle 138 represents green.

For any two points on the chromaticity diagram 100, all colors that can be formed by mixing the two colors represented by the two points lie on a straight line connecting the two points. If a given filter is progressively inserted into a collimated, unfiltered light beam, the resulting CIE xy point for the light moves in a straight line away from the CIE xy point for the unfiltered light. The movement of the xy point continues to move in a straight line until the given filter is totally inserted. Directions of the circles 130, 134 and 138 from the xenon light chromaticity 110 provide indications as to how light from the subject xenon source is filterable to provide the tints shown in Table 1. For example, if it is desirable to provide the safety-filtered xenon light represented by the square 110 with a yellow tint represented by the circle 130, a filter may be configured to cause the xy point for the light 110 to move toward the circle 130. Such movement is toward a spectral region somewhere between red and magenta and away from a spectral region that includes green. This movement can be effected by removing light from the spectral region that includes green.

If a filter is configured to block a small region of the spectrum, xy coordinates for the lamp color on the diagram 100 move away from this spectral region when the filter is inserted. As a blocked region broadens, distance of the shift of xy coordinates increases on the diagram 100. Accordingly, in various implementations a filter may be designed by evaluating the location and width of a blocking region for the filter to obtain a desired color tint.

An exemplary transmission curve for a blocking region to provide a yellow tint for safety-filtered xenon light is indicated generally in FIG. 4 by reference number 200. A blocking region 204 is centered at about a 520-nanometer (i.e., a green) wavelength. In some implementations, transitions between a blocking region and transmission regions may tend to be sharp rather than gradual, as exemplified by a transition 208 between the blocking region 204 and transmission regions 212.

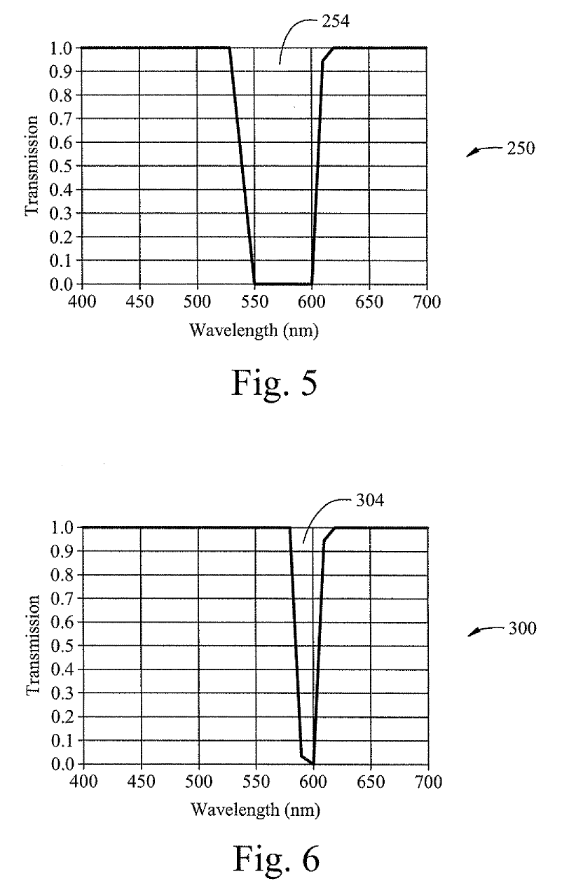

An exemplary transmission curve for a blocking region to provide a cyan tint for safety-filtered xenon light is indicated generally in FIG. 5 by reference number 250. A blocking region 254 is centered at about a 575-nanometer (i.e., a yellow) wavelength.

An exemplary transmission curve for a blocking region to provide a green tint for safety-filtered xenon light is indicated generally in FIG. 6 by reference number 300. A blocking region 304 is centered at about a 590-nanometer (i.e., an orange) wavelength.

In some filter configurations, partial blocking may be provided that extends over a spectral region wider than those shown in FIGS. 4-6. One or more transitional curves may be determined for a spectral blocking region and filtering may be provided for the spectral region in accordance with the transitional curve(s). Such a curve may be easier to configure in a given filter than, for example, the comparatively sharp transitions 208 between transmission and blocking regions as shown in FIG. 4.

Inserting a filter into a light beam generally reduces luminance. In some implementations of the disclosure, filters may be designed to exhibit luminance as shown in Table 2. A luminance factor of 1 would imply no loss of luminance. Actual luminance values in particular filters may differ from those shown in Table 2, dependent, e.g., on how close to 100 percent transmission can be achieved away from a blocking region and on efficiency of any antireflection coatings that may be provided.

TABLE 2

| Filter | Blocking range | Luminance factor |
|---|---|---|
| Yellow | 502-526 nm | 0.86 |
| Cyan | 540-606 nm | 0.45 |
| Green | 585-605 nm | 0.88 |

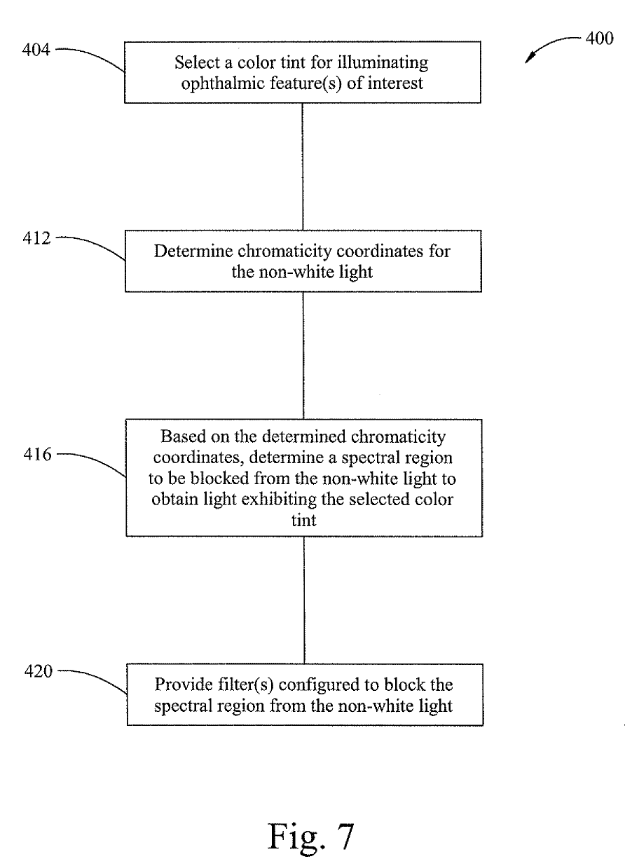

One implementation of a method of providing color-tinted light in an ophthalmic illumination system configured to produce non-white light is indicated generally in FIG. 7 by reference number 400. The method 400 can be used to provide one or more filters that take into account the spectrum of a non-white light. In process 404 a color tint for illuminating one or more ophthalmic features is selected. The color tint may, e.g., have coordinates the same as or similar to one of the tints described in Table 1. In process 412 chromaticity coordinates for the non-white light are determined. In process 416, a spectral region is determined based on the determined chromaticity coordinates. The spectral region is to be blocked from the non-white light to obtain light exhibiting the selected color tint. In process 420 one or more filters are provided that are configured to block the spectral region from the non-white light.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A method of providing color-tinted light in an ophthalmic illumination system having a light source configured to produce non-white light, the method comprising:
selecting a color tint for illuminating one or more ophthalmic features of interest;
determining chromaticity coordinates for the non-white light;
based on the determined chromaticity coordinates, determining a spectral region to be blocked from the non-white light to obtain light from the light source exhibiting the selected color tint; and
providing the illumination system with one or more filters configured to block the spectral region from the non-white light.

2. The method of claim 1, wherein the light source is a xenon light source configured to produce safety-filtered light, the method further comprising blocking from the safety-filtered light a spectral region away from which a line generally points from a mapping on a chromaticity diagram of the safety-filtered light toward a mapping on the diagram of the selected color tint.

3. The method of claim 1, further comprising:
determining one or more transitional curves for the spectral region; and
filtering the spectral region in accordance with the one or more transitional curves.

4. The method of claim 1, comprising selecting yellow as the color tint.

5. The method of claim 1, wherein light exhibiting the selected color tint is described by International Commission on Illumination (CIE) xy coordinates of (CIE x=0. 3534, CIE y=0.3619).

6. The method of claim 1, comprising selecting cyan as the color tint.

7. The method of claim 1, wherein light exhibiting the selected color tint is described by CIE xy coordinates of (CIE x=0.2462, CIE y=0.2929).

8. The method of claim 1, comprising selecting green as the color tint.

9. The method of claim 1, wherein light exhibiting the selected color tint is described by CIE xy coordinates of (CIE x=0.2954, CIE y=0.3901).

10. The method of claim 1, further comprising filtering selected end portions of the spectrum from light from the light source to produce the non-white light.

11. A method of providing color-tinted light in an ophthalmic illumination system having a xenon light source configured to produce safety-filtered light, the method comprising:
selecting a color tint for illuminating one or more ophthalmic features of interest;
determining chromaticity coordinates for the selected color tint;
determining chromaticity coordinates for the safety-filtered light;
using the determined coordinates to locate a spectral region to be blocked from the safety-filtered light to obtain light exhibiting the selected color tint; and
providing the illumination system with one or more filters configured to filter frequencies in the located spectral region from the safety-filtered light.

12. The method of claim 11, wherein the selected color tint is yellow, and the spectral region is centered at about a 520-nanometer wavelength.

13. The method of claim 11, wherein the selected color tint is cyan, and the spectral region is centered at about a 575-nanometer wavelength.

14. The method of claim 11, wherein the selected color tint is green, and the spectral region is centered at about a 590-nanometer wavelength.

15. The method of claim 11, comprising blocking from the safety-filtered light a spectral region away from which a line generally points from a mapping on a chromaticity diagram of the safety-filtered light toward a mapping on the diagram of the selected color tint.

16. An ophthalmic illumination system having a light source and a light probe configured to receive light from the light source via an optical light path, the illumination system comprising one or more filters configured to cause non-white light from the light source to exhibit a selected color tint;
at least one of the one or more filters configured to block from the non-white light a spectral region away from which a chromaticity of the non-white light is changeable to cause the light to exhibit a chromaticity of the selected tint.

17. The ophthalmic illumination system of claim 16, the light source comprising a safety-filtered xenon lamp.

18. The ophthalmic illumination system of claim 17, wherein the selected color tint is yellow, and the spectral region is centered at about a 520-nanometer wavelength.

19. The ophthalmic illumination system of claim 17, wherein the selected color tint is cyan, and the spectral region is centered at about a 575-nanometer wavelength.

20. The ophthalmic illumination system of claim 17, wherein the selected color tint is green, and the spectral region is centered at about a 590-nanometer wavelength.
